(19) United States
(12) Patent Application Publication
(10) Pub. No.: US 2021/0031055 A1
(43) Pub. Date: Feb. 4, 2021

(54) MRI ADAPTATION FOR RADIOTHERAPY MACHINES

(71) Applicants: Board of Regents of the University of Texas System, Austin, TX (US); Rensselaer Polytechnic Institute Office Of Technology Transfer, Troy, NY (US)

(72) Inventors: Steve Bin Jiang, Dallas, TX (US); Xun Jia, Dallas, TX (US); Ge Wang, Loudonville, NY (US); Hak Choy, Dallas, TX (US); Nima Hassan-Rezaeian, Austin, TX (US); Chenyang Shen, Dallas, TX (US)

(21) Appl. No.: 17/045,978

(22) PCT Filed: Apr. 11, 2019
(86) PCT No.: PCT/US2019/027072

§ 371 Date: Oct. 7, 2020

Related U.S. Application Data
(60) Provisional application No. 62/655,923, filed on Apr. 11, 2018.

(51) Int. Cl. A61N 5/10; G01R 33/3815; A61B 6/04; A61B 5/055; G01R 33/38; G01R 33/385; G01R 33/48; G01R 33/3875

(52) U.S. Cl. CPC A61N 5/1049; A61N 5/1081; G01R 33/3815; A61B 6/0407; A61B 5/0555; G01R 33/3806; A61N 2005/1055; A61N 5/1083; G01R 33/3802; G01R 33/4808; G01R 33/3875; A61N 2005/1061; A61N 2005/1094; G01R 33/385

(57) ABSTRACT

Various examples of methods, systems, apparatus and devices are provided for MRI adaptation for radiotherapy machines. In one example, a system for MRI-guided radiotherapy can include a mounting ring and superconducting magnets. The mounting ring can be installed on a gantry of a LINAC to rotate about an isocenter of the LINAC moving with the gantry. The first and second superconducting magnet can be positioned substantially parallel to each other at a separation distance with the centers substantially aligned. The first and second superconducting magnets can provide a main magnetic field within a region of interest located between the first and second superconducting magnets. The superconducting magnets can have an aperture positioned at the center of each magnet and can allow a radiotherapy beam emitting from the gantry head to pass through the apertures. In another example, superconducting magnets can be installed at opposite ends of a LINAC gantry.

### CROSS-REFERENCE TO RELATED APPLICATION

This application claims the benefit of and priority to U.S. Provisional Application No. 62/655,923 entitled, “ON-BOARD INTERIOR MRI,” filed on Apr. 11, 2018, the contents of which being incorporated by reference herein in its entirely.

### BACKGROUND

Image guidance plays a critical role in radiotherapy to ensure treatment accuracy. Cone-beam computer tomography (CBCT) installed on a medical linear accelerator (LINAC) is routinely used in clinics for this purpose. While CBCT can provide an x-ray attenuation image to guide patient positioning, low soft-tissue contrast affects the delineation of anatomical features, hindering setup accuracy in many cases.

### SUMMARY

Provided herein is a system for MRI-guided radiotherapy that can include a mounting ring configured to be installed on a gantry of a linear accelerator (LINAC) and configured to rotate about an isocenter of the LINAC moving with the gantry; a first superconducting magnet connected to the mounting ring, the first superconducting magnet positioned in a first plane contacting a gantry head of the LINAC; a second superconducting magnet connected to the mounting ring, the second superconducting magnet positioned in a second plane substantially parallel to the first plane at a separation distance, a center of the second superconducting magnet substantially aligned with a center of the first superconducting magnet; where the first and second superconducting magnets are configured to provide a main magnetic field within a region of interest, the region of interest located between the first superconducting magnet and the second superconducting magnet; and where each of the first and second superconducting magnets have an aperture between an inner surface facing the isocenter and an outer surface facing away from the isocenter, each aperture positioned at the center of each superconducting magnet and configured to allow a radiotherapy beam emitting from the gantry head to pass through the apertures. In various examples, the main magnetic field produced by the first and second superconducting magnets do not substantially interfere with operation of the LINAC. A measurement of magnetic field near an accelerating waveguide of the LINAC can be less than 5 Gauss. A measurement of magnetic field near the gantry head of the LINAC can be less than 400 Gauss. A measurement of homogeneity (ΔB₀/B₀) for magnetic field within the region of interest can be less than 50 ppm. In some embodiments, the gantry head of the LINAC is shielded from magnetic fields. The mounting ring can be further configured to provide shielding from at least one of: magnetic fields, x-rays, or photons produced by the LINAC. The system for MRI-guided radiotherapy can include a magnetic resonance detector configured to collect excitation signal data to generate a magnetic resonance image. The mounting ring can be further configured to mount an x-ray imaging system, where the x-ray imaging system configured to operate in a plane perpendicular to the radiotherapy beam of the LINAC, the x-ray imaging system including: an x-ray source attached to the mounting ring and configured to direct an x-ray beam toward the region of interest; and an x-ray detector attached to the mounting ring opposite the x-ray source, and where the magnetic fields produced by the first and second superconducting magnets do not interfere with operation of the x-ray imaging system. In some examples, the x-ray detector is configured to collect x-ray data to generate an x-ray tomographic image of the region of interest. In some examples, the x-ray data and the excitation signal data are collected simultaneously with rotation of the LINAC gantry. In various examples, the region of interest can be within a body of a patient, the patient being positioned on a patient couch to receive radiotherapy treatment. The system for MRI-guided radiotherapy can also include at least one computing device; and program instructions executable in the at least one computing device that, when executed by the at least one computing device, cause the at least one computing device to: collect data from excitation signals to generate a magnetic resonance image containing the region of interest; and apply a regularization transformation to portion of image containing the region of interest.

Also provided herein is a system for magnetic resonance imaging that can include a first superconducting magnet positioned in a first plane; and a second superconducting magnet positioned in a second plane substantially parallel to the first plane at a separation distance, a center of the second superconducting magnet substantially aligned with a center of the first superconducting magnet; and where the first and second superconducting magnets are configured to provide a main magnetic field within a region of interest, the region of interest located between the first superconducting magnet and the second superconducting magnet. In various examples, each of the first and second superconducting magnets can include super conducting coils. In various examples, each of the super conducting coils of the first and second superconducting magnets can include a plurality of superconducting fibers, the plurality of superconducting fibers configured to receive liquid helium. The system for magnetic resonance imaging can also include a plurality of coils configured to generate gradient magnetic fields along x, y, and z directions of an orthogonal coordinate system. In some examples, the first and second superconducting magnets can be positioned parallel to an x-z plane; a first y-gradient coil positioned within the first superconducting magnet, the first y-gradient coil configured to provide a magnet field along the y-direction, the y-direction perpendicular to the x-z plane; a first x-gradient coil positioned on an inner surface of the first superconducting magnet facing the region of interest, the first x-gradient coil configured to provide a magnet field along the x-direction; and a first z-gradient coil positioned on the inner surface of the first superconducting magnet facing the region of interest, the first z-gradient coil configured to provide a magnet field along the z-direction. The gradient data can be collected to generate a volumetric image. Each of the first and second superconducting magnets can have an aperture between an inner surface facing the region of interest and an outer surface facing away from the region of interest, each aperture positioned at the center of each superconducting magnet and can be configured to allow a radiotherapy beam to pass through the apertures. The first superconducting magnet can be positioned between a radiotherapy source and the region of interest and the second superconducting magnet is positioned between the region of interest and an imaging device. The region of interest can be within a body of a patient. The main magnetic field (B₀) can be approximately 0.2 to 0.8 Tesla within the region of interest. The separation distance can be approximately 50 to 90 cm. The region of interest can be within a substantially spherical region having a diameter of approximately 10 to 20 cm. The system for magnetic resonance imaging can include a magnetic resonance detector configured to collect excitation signals to produce an image. The system for magnetic resonance imaging can be configured to be mounted on a robotic arm. The system for magnetic resonance imaging can be configured to rotate about an axis.

Also provided herein is a retrofit MRI assembly that can include a main magnet comprising spatially separated first and second portions, the first portion including a first set of superconducting wires disposed within a first circular housing concentric about an isocenter of a gantry of a circular radiation therapy machine, the first circular housing installed at a first end exterior to the gantry; the second portion comprising a second set of superconducting wires disposed within a second circular housing concentric about the isocenter of the gantry, the second circular housing installed at a second end exterior to the gantry of the circular radiation therapy machine, where the second set of superconducting wires are substantially parallel to the first set of superconducting wires with a center of the second set of superconducting wires substantially aligned with a center of the first set of superconducting wires, the first and second sets of superconducting wires separated by at a gantry separation distance; shimming coils; and gradient coils; where the first set and the second set of superconducting wires can be configured to provide a main magnetic field within a region of interest located at the isocenter of the gantry. The retrofit MRI assembly can include a third set of circular superconducting wires positioned at the first end of the gantry and a fourth set of circular superconducting wires positioned at the second end of the gantry, wherein the third set and the fourth set are concentric with the first set and the second set of superconducting wires, and wherein the third set and the fourth set are located at a smaller radii from the isocenter than the first set and the second set of superconducting wires. The main magnet can be configured to produce a magnetic field that does not interfere with an operation of a LINAC of the circular radiation therapy machine. When energized, a measurement of magnetic field near an accelerating waveguide of a LINAC of the circular radiation therapy machine can be less than 5 Gauss. When energized, a measurement of magnetic field during operation near a gantry head of a LINAC can be less than 400 Gauss. A measurement of homogeneity (ΔB₀/B₀) for a magnetic field within the region of interest can be less than 50 ppm. In various examples, a gantry head of a LINAC can be shielded from magnetic fields. The first circular housing can include a cryostat housing and the second circular housing comprise a cryostat housing. The retrofit MRI assembly can include a magnetic resonance detector configured to collect excitation signal data to generate a magnetic resonance image. The circular radiation therapy machine can include a LINAC and an x-ray imaging system, the x-ray imaging system configured to operate in a plane perpendicular to a radiotherapy beam of the LINAC. In various examples, the main magnetic field produced by the first and the second sets of superconducting wires does not interfere with operation of the x-ray imaging system. The x-ray imaging system can be configured to collect x-ray data to generate an x-ray tomographic image of the region of interest. The retrofit MRI assembly can include at least one computing device; and program instructions executable in the at least one computing device that, when executed by the at least one computing device, cause the at least one computing device to: generate a magnetic resonance image containing a field of view based upon data collected from excitation signals; and apply a regularization transformation to a portion of image containing the field of view.

Also provided herein is a system for magnetic resonance imaging that can include a first superconducting magnet positioned in a first plane; and a second superconducting magnet positioned in a second plane substantially parallel to the first plane at a gantry separation distance, a center of the second superconducting magnet substantially aligned with a center of the first superconducting magnet; and where the first and second superconducting magnets are configured to provide a main magnetic field within a region of interest, the region of interest located between the first superconducting magnet and the second superconducting magnet. Each of the first and second superconducting magnets can include super conducting coils. Each of the super conducting coils of the first and second superconducting magnets can include a plurality of superconducting fibers, the plurality of superconducting fibers can be configured to receive liquid helium. The system for magnetic resonance imaging can include a plurality of coils configured to generate gradient magnetic fields along x, y, and z directions of an orthogonal coordinate system. The system for magnetic resonance imaging can include a gradient field generator comprising a plurality of layers of self-shielded gradient coils and a plurality of layers of shimming coils. Gradient data can be collected to generate a volumetric image. The main magnetic field (B₀) can be approximately 0.4-0.7 Tesla within a region of interest.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 illustrates main components of an iMRI device, according to various embodiments of the present disclosure.

FIGS. 2A-2D illustrate the iMRI device mounted on a LINAC gantry at different gantry and couch angle combinations, according to various embodiments of the present disclosure.

FIG. 3 illustrates an example of the iMRI device installed on a mounting ring with the mounting ring attached to a LINAC, according to various embodiments of the present disclosure.

FIG. 4 illustrates an example of a mounting ring including the iMRI device and a CBCT imaging system positioned perpendicular to it, according to various embodiments of the present disclosure.

FIG. 5 illustrates the spatial distribution of they (top) and x (bottom) component of the B₀ filed in the x-y plane, according to various embodiments of the present disclosure.

FIG. 6A illustrates voxels excited by an RF pulse corresponding to the slice at z₀=0 inside the ROI of diameter 15 cm (sphere), according to various embodiments of the present disclosure.

FIGS. 6B and 6C illustrate an MRI image M(x,y,z₀) at the slice intended to be selected and an actual signal M(x,y) of a region of interest (ROI), according to various embodiments of the present disclosure.

FIG. 7 illustrate results reconstructed using conventional FBP (left) and method described herein (right) using 360, 180, and 120 projections respectively, according to various embodiments of the present disclosure.

FIG. 8 illustrates examples of existing circular radiation therapy machines, where the outermost shell may be removed, according to various embodiments of the present disclosure.

FIG. 9 illustrates a front view of example circular radiation therapy machine with LINAC and CBCT systems mounted on the ring gantry, according to various embodiments of the present disclosure.

FIG. 10 illustrates an example desired magnetic field intensity and uniformity within a 40 cm diameter field of view at the center of an example existing circular radiation therapy machine, according to various embodiments of the present disclosure.

FIG. 11 illustrates an example Retrofit MRI assembly, according to various embodiments of the present disclosure.

FIG. 12 illustrates a front or rear view of the example system of FIG. 11, according to various embodiments of the present disclosure.

FIG. 13 illustrates a cutaway perspective view of the example system of FIG. 11, according to various embodiments of the present disclosure.

FIG. 14 illustrates a side view of the example system of FIG. 11, according to various embodiments of the present disclosure.

FIG. 15 illustrates a cutaway side view of the example system of FIG. 11, according to various embodiments of the present disclosure.

FIG. 16 illustrates a perspective view of an example Retrofit MRI assembly mounted on an example existing circular radiation therapy LINAC machine, with the outer shell removed, according to various embodiments of the present disclosure.

FIG. 17 illustrates a cutaway perspective view of the example Retrofit MRI assembly mounted on the example existing circular radiation therapy machine of FIG. 16, according to various embodiments of the present disclosure.

FIG. 18 illustrates an enlarged cutaway view of the superconducting wires and their example cryostat housing for an example main magnet, according to various embodiments of the present disclosure.

FIG. 19 illustrates the layers of an example cryostat housing for superconducting wires, according to various embodiments of the present disclosure.

FIG. 20 illustrates a front view of the example Retrofit MRI assembly mounted on the example existing circular radiation therapy machine of FIG. 16, according to various embodiments of the present disclosure.

FIG. 21 illustrates a cutaway front view of the example Retrofit MRI assembly mounted on the example existing circular radiation therapy machine of FIG. 16, according to various embodiments of the present disclosure.

FIG. 22 illustrates a cutaway perspective view from the rear of the example Retrofit MRI assembly mounted on the example existing circular radiation therapy machine of FIG. 16.

FIG. 23 illustrates a cutaway side view of the example Retrofit MRI assembly mounted on the example existing circular radiation therapy machine of FIG. 16.

FIGS. 24A-24C illustrate the mechanical details of different example components of a Retrofit MRI assembly, according to various embodiments of the present disclosure.

FIG. 25 illustrates an example gradient field generator used in the example Retrofit MRI assemblies, according to various embodiments of the present disclosure.

FIG. 26A and 26B illustrates example RF sender coils used in the example Retrofit MRI assemblies, according to various embodiments of the present disclosure.

FIG. 27A and 27B illustrate example already-available RF receiver coils used in the example Retrofit MRI assemblies, according to various embodiments of the present disclosure.

FIG. 28 illustrates an example method of designing or making a Retrofit MRI assembly for different geometries of pre-existing circular radiation therapy machines, according to various embodiments of the present disclosure.

FIG. 29 illustrates an example of the spatial distribution of the B₀ field and iso-surfaces generated by the main magnet, according to various embodiments of the present disclosure.

### DETAILED DESCRIPTION

Disclosed herein are various examples of methods, systems, apparatus and devices related to an MRI adaptation for existing radiotherapy machines to provide an interior tomography approach for MRI-guided radiation therapy. Although both open and circular ring LINAC systems are shown as existing equipment for reference implementations, the MRI adaptation described herein is not limited to LINAC systems and can be adapted to other radiotherapy machines. Reference will now be made in detail to the description of the embodiments as illustrated in the drawings, wherein like reference numbers indicate like parts throughout the several views.

Several MRI-LINAC systems have been developed to combine a full diagnostic Magnetic Resonance Imaging (MRI) scanner with a radiotherapy machine to address setup accuracy. Described herein is a new concept for the development of the MRI-LINAC system. Instead of combining a full MRI scanner with the LINAC, an interior MRI (iMRI) can be used to image a specific region of interest (ROI) containing the radiation treatment target. The iMRI can provide local imaging of high soft-tissue contrast for tumor delineation. Meanwhile, megavoltage CBCT currently available on the LINAC can be used to deliver a global image of the patient's anatomy. The embodiments described herein provide for an iMRI system design and its integration to an LINAC platform, with consideration of magnetic field design and imaging capability.

In cancer radiotherapy, it is important to precisely deliver an amount of radiation dose to the cancerous target tumor, while sparing nearby normal tissues and organs to avoid harming them. Image guidance is an important step to ensure treatment accuracy. Before a treatment delivery, Image Guided Radiation Therapy (IGRT) first acquires a scan of the patient's anatomy on the day of treatment. The patient is then accurately positioned based on the internal anatomy with respect to the radiotherapy beam as designed in the treatment plan. Modern radiotherapy approaches have made image guidance increasingly important. For instance, the use therapeutic delivery methods such as intensity modulated radiation therapy (IMRT), as well as treatment modalities such as proton and heavy ion therapy, have enabled dose distributions that are conformal to the target, but at the same time, vulnerable to positioning errors. In these scenarios, a small spatial misalignment between the target and the radiotherapy beam could potentially cause a drop in tumor coverage and/or harm normal tissue near the tumor.

Over the years, kilo-voltage cone-beam computer tomography (CBCT) installed on a medical linear accelerator (LINAC) has evolved to be a widely used image-guidance tool in radiotherapy. While its value in terms of ensuring setup accuracy have been repeatedly demonstrated by many studies, the predominant role of CBCT in IGRT has been challenged by Magnetic Resonance Imaging (MRI) due to the advantages of superior image contrast, the absence of ionizing radiation, and the potential of functional imaging. Yet, MRI-guided radiation therapy (MRgRT) requires the integration of an MRI scanner on a LINAC platform. This is a challenging engineering problem because of the sharp conflicts between the two devices in terms of physics and geometry. First, a strong magnetic field needed by the MRI affects many electronic components inside the LINAC that are susceptible to electromagnetic interference. Second, a conventional MRI system employs a bulky and complex design to realize a sufficiently large field of view (FOV), hindering integration into a space-limited LINAC platform.

Despite the challenges, tremendous progress has been made by many groups towards the integration of MRI with a radiotherapy machine. Nonetheless, attempts to resolve electromagnetic interference between MRI and LINAC under a tight geometry constraint led to systems with suboptimal or compromised functions at an increased cost. For instance, the commercially available system from ViewRay Inc. combined a low-field (0.3 T) MRI and a low-energy Co-60 therapy machine, which is not ideal for treating deeply seated tumors. In the Elektas prototype system, and in other similar systems integrating a 1.5 T MRI and a LINAC, have a bulky design that prohibited LINAC couch rotation. This reduced the freedom to develop a high quality treatment plan in some cases, e.g., head-and-neck tumors and stereotactic body radiotherapy. Moreover, all the existing MRI-LINAC systems employed specifically designed LINACs and were incompatible with traditional LINACs. Clinical adoption of these systems has to absorb a high cost burden of the new system development and facility deployment. For those clinics that already have LINACs installed, purchasing a new and expensive MRI-LINAC is a particular concern.

Existing efforts have exclusively focused on combining a full diagnostic MRI system with a radiotherapy machine. Recently, advancements in the interior tomography field have enabled theoretically exact and numerically stable reconstruction of an image in an interior region of interest (ROI). The ROI can be made small but sufficient for clinical applications. Motivated by this advancement, described herein is a new concept for the development of an MRI adapted for existing radiotherapy machines, such as a LINAC system. Instead of combining a full MRI scanner and a LINAC, an interior MRI (iMRI) approach can be employed that images a local ROI of the most radio-therapeutic relevance, aided by a megavoltage (MV) CBCT image as a complementary global anatomical prior. In this approach, the small ROI will only need a homogeneous magnetic field just enough to cover it. Hence, technical demands on hardware and compatibility with an LINAC could be relaxed to achieve a compact MRI design that can be geometrically and electromagnetically compatible with the current LINAC systems. Such an iMRI system can be retrofitted to any LINAC system to enable MRgRT.

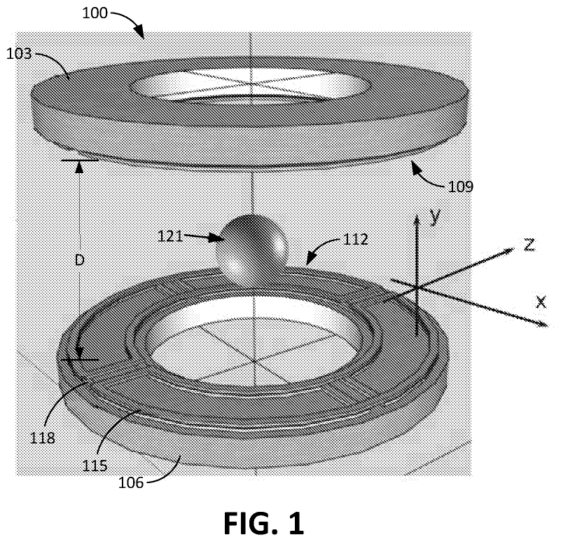

In an embodiment, an MRI adaptation can include the main hardware components for the iMRI system 100, illustrated in FIG. 1, can include top and bottom superconducting magnets 103,106 configured to provide the main field B₀ of about 0.5 T inside the imaging region of interest (ROI) or field of view (FOV) 121 having a diameter of about 15 cm. The separation (D) between the two magnets can be ˜70 cm, which is configured to provide enough space to accommodate a typical patient (not shown). This can be achieved by designing the current pattern within the superconducting coil systems using standard optimization techniques. A superconducting magnet typically contains cables made of a superconducting material. The cable has to be cooled to below the critical temperature to maintain its superconducting state. Conventionally, this is achieved through the use of a cryostat. The big size and complexity impede the integration of the MRI device to an LINAC gantry. Embodiments of the iMRI system are configured to use superconducting fibers as an alternative to construct the magnet. These superconducting fibers are fabricated to contain a space allowing injection of liquid helium. After injection, fibers can be maintained in the superconducting state for an extended time period for MRI data acquisition, before being heated to the critical temperature. This property is important in terms of achieving a light weighted iMRI system suitable for mounting to the LINAC gantry, as it is configured to eliminate the need for a cryostat in the superconducting magnet.

For volumetric imaging purpose, the iMRI system can also contain coils to generate gradient magnetic fields along x, y, and z directions. The y gradient can be formed using gradient coils located inside in the superconducting magnets 103,106. At the inner facing surfaces 109,112 of the two superconducting magnets 103,106 are x-gradient coils 115 and z-gradient coils 118 configured to provide magnetic fields with a constant gradient along the x and z directions, respectively. The general placement of coils are shown in FIG. 1 to illustrate that two gradient fields are achievable, as has been demonstrated in commercially available open MRI systems. The exact wire winding pattern suitable for iMRI imaging can be configured and designed following standard techniques.

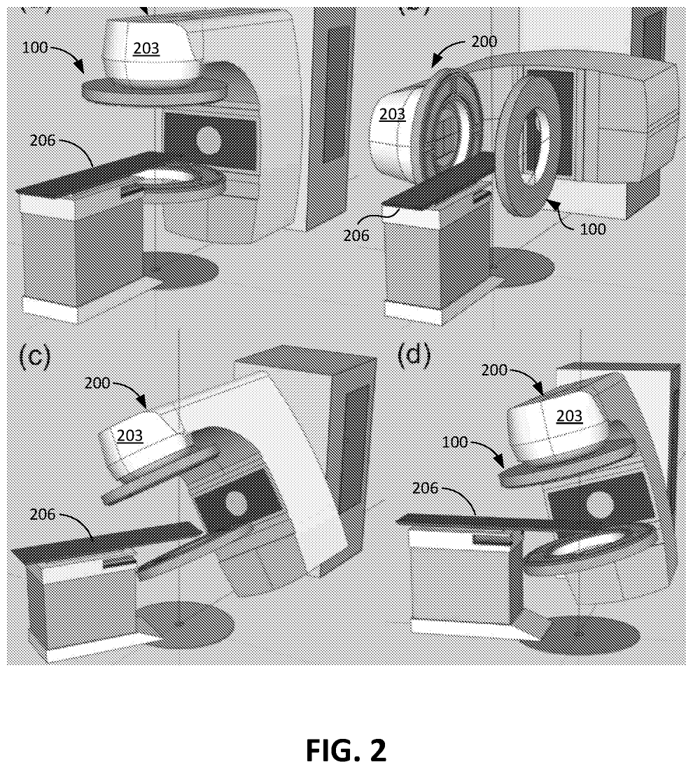

As illustrated in FIG. 2A, the iMRI system 100 can have a compact design, such that the iMRI system 100 can be mounted on the LINAC gantry 203. The holes on the superconducting magnets allow the radiotherapy beam to pass through. Hence, the electronic portal imaging device (EPID) available on the current LINAC 200 can still receive photon beams from the LINAC, which allows acquisition of portal images. After a full gantry rotation, this setup enables MV CBCT data acquisition to obtain a global view of the patient anatomy complementing the interior MR image inside the ROI.

Due to the compact design, this system still allows radiotherapy treatments conducted at different combinations of the gantry 203 and the couch 206 angles, preserving non-coplanar radiotherapy treatment delivery to a large extent. This is a desired feature, as non-coplanar treatments are advantageous in many cases in terms of reducing normal tissue doses. Three examples of system geometry with different gantry 203 and couch 206 angle combinations are illustrated in FIGS. 2B-2D.

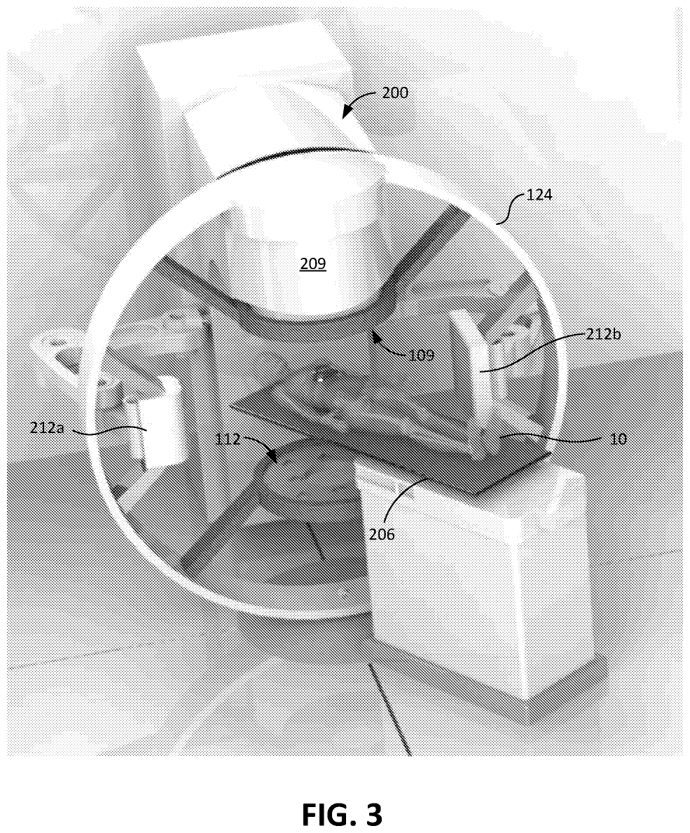

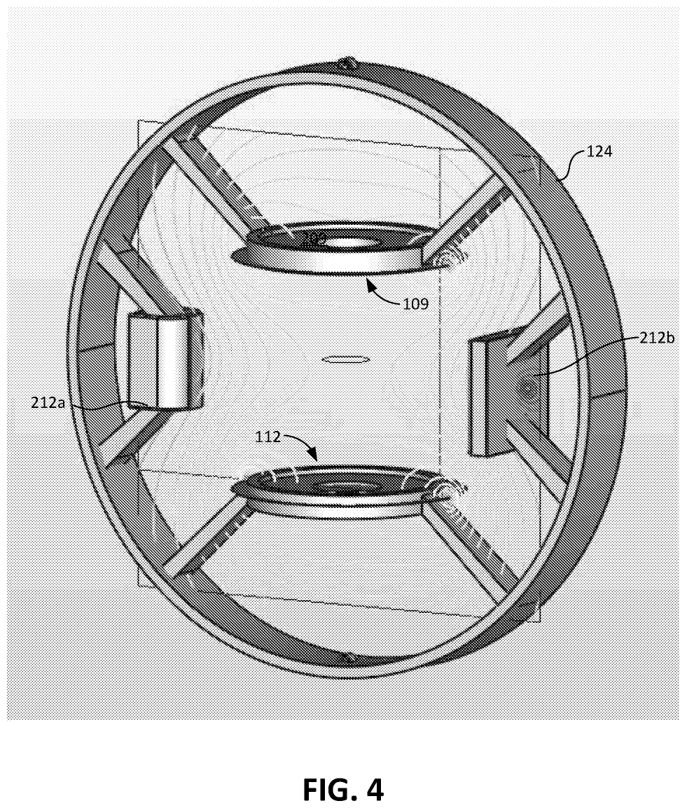

The system 100 can be mounted on a positioning ring 124 to maintain the position of the superconducting magnets 103,106 with respect to the gantry head 209 of the LINAC 200 and the patient couch 206, as shown in FIG. 3. The positioning ring 124 can also provide mounts for a CBCT x-ray imaging system 212a,212b. The iMRI 100 and CBCT 212 can be positioned substantially perpendicular to each other and configured such that interference from each system is minimized, as shown in FIG. 4.

The iMRI system 100 can perform data acquisition similar to a standard MRI system. However, the homogeneous main field B₀ that exists only in the small ROI creates an additional issue that has to be considered. A standard slice selection technique can be used by applying a slice selection z-gradient field. The other two gradient fields will be used for frequency encoding and phase encoding. For example, considering a slice orthogonal to the z axis with a coordinate z=z₀, a radio frequency (RF) pulse with frequency f₀=γ(B₀+z₀G_{z}) should be used, where G_{z} is the gradient amplitude. However, this pulse will in fact excite all points in a set Ω={f(x, y, z):γ[B₀(x, y, z)+zG_{z}]=f₀}, not only the targeted slice inside the ROI. Hence, at the moment of measurement, the signal after demodulation is

$$\begin{matrix}S ( {k}_{x} , {k}_{y} ) = {\int }_{Ω} dxdydzM ( x , y , z ) {e}^{- i ( {k}_{x} x + {k}_{y} y )} , \\ = \int d x d y {e}^{- i ( {k}_{x} x + {k}_{y} y )} {\int }_{{Ω}_{( x , y )}} d z M ( x , y , z ) , \\ = \int d x d y {e}^{- i ( {k}_{x} x + {k}_{y} y )} \hat{M} ( x , y ) .\end{matrix} \qquad ( 1 )$$

where M(x,y,z) is the 3D magnetization distribution. Ω_{(x,y)} is a subset of Ω, namely the intersection between Ω and a straight line that is parallel to the z axis and passing through the point (x,y,z₀). This indicates that the measured signal is equivalently generated from an 2D image \hat{M}_{(x,y)}=∫_{(x,y)}dzM(x,y,z). This indeed creates an issue that needs special attention. For a coordinate (x,y) that is inside the ROI, Ω_{(x,y)} certainly contains the point (x,y,z₀) due to the set up with a homogeneous B₀ field and a slice selection gradient field. Hence, the measured signal contains contributions from the selected slice at z₀ inside the ROI. However, if Ω_{(x,y)} also contains points with other z coordinates, the integration along the z axis will mix signals at those z coordinates with that at z₀. In this case, the targeted signal cannot be easily distinguished from other mixed signals, deteriorating image accuracy inside the ROI. This problem can be avoided by carefully designing the magnetic field B₀, such that the aforementioned condition is not satisfied and hence \hat{M}(x,y)=M(x,y,z₀) inside the ROI.

With the measurement S(kₓ,k_{y}) made, standard reconstruction techniques using analytical reconstruction or iterative reconstruction techniques apply. For radiotherapy online imaging applications, data acquisition time is a concern. Therefore, k-space data undersampling is desired to speed up the data acquisition process. In this case, iterative reconstruction will be advantageous, as analytical reconstruction techniques are more vulnerable to image artifacts caused by the data undersampling.

Let us represent the magnetization by a vector u. Discretizing Eq. (1) arrives at a linear equation:

AFu=g, (2)

where F is the Fourier transform operator, A is an undersampling operator corresponding to the sampled k-space locations, and g is a vector containing the measurement data. Since the solution u represents a 2D image, a certain type of image regularization can be applied to constrain the solution. As an example, tight frame (TF) can be used as a regularization transformation and solve the problem:

$${min}_{u} {\vert Wu \vert}_{1} , s . t . AFu = ℊ , \qquad ( 3 )$$

where W is a TF transform operator. Minimizing the I₁ norm of the transformed image Wu inherently assumed that the solution image u has a sparse representation under the TF transformation. Note that in the solution image, only the region inside the ROI is of interest. Hence, only apply the regularization inside the ROI is applied. This optimization problem (3) can be efficiently solved using the alternating direction method of multipliers.

Simulation studies were performed to further demonstrate a magnet design. Specifically, an inverse optimization problem was solved with respect to the current pattern inside the two superconducting magnets. The objective function penalized deviation of the magnetic field from the targeted homogeneous field B₀=0.5 T throughout the FOV. A hard constraint was also imposed to ensure the field strength at the LINAC gantry head is tolerable. After that, a volumetric MRI image of a liver cancer patient was selected. For a slice of interest, the set was first computed and then the acquired signal was synthesized according to Eq. (1).

For the purpose of proof-of-principle, only undersampling along a number of equiangular straight lines passing through the k-space origin was considered. This is also known as projection data acquisition. With the synthesized data, the image was then reconstructed via the model in Eq. (3). For comparison purpose, reconstruction using the conventional filtered backprojection (FBP) algorithm was also performed. Finally, the image quality was evaluated by comparing with the ground truth input MRI image.

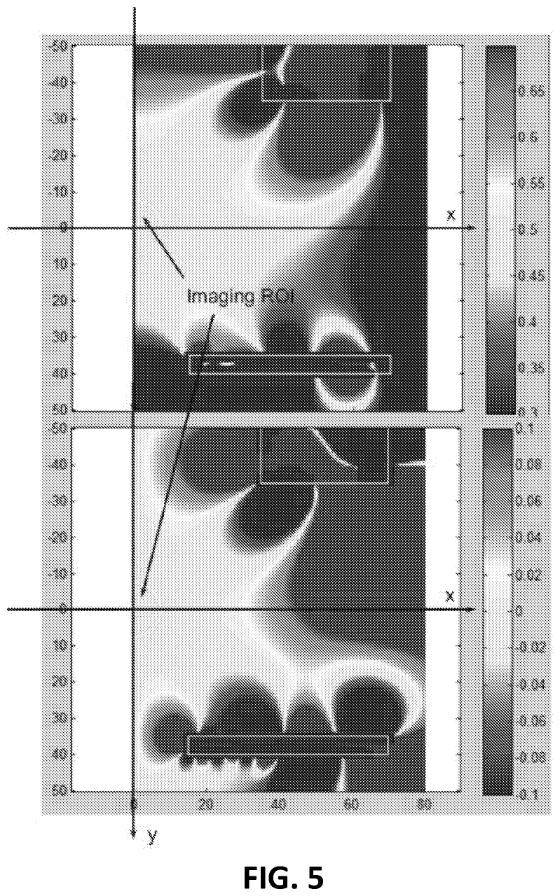

FIG. 5 presents the spatial distribution of the x and y component of the B₀ field in the x-y plane. The component perpendicular to this plan is zero. The field in 3D space is rotationally symmetric about the y axis. Using the current optimization technique, the B₀ field was made homogeneous inside the FOV of a diameter of 15 cm with ΔB₀/B₀ about 50 ppm. In addition, at the positive direction y about 50 cm locates the gantry head of a LINAC. The field at this location was constrained to less than 400 Gauss, which was expected to be tolerable by a LINAC.

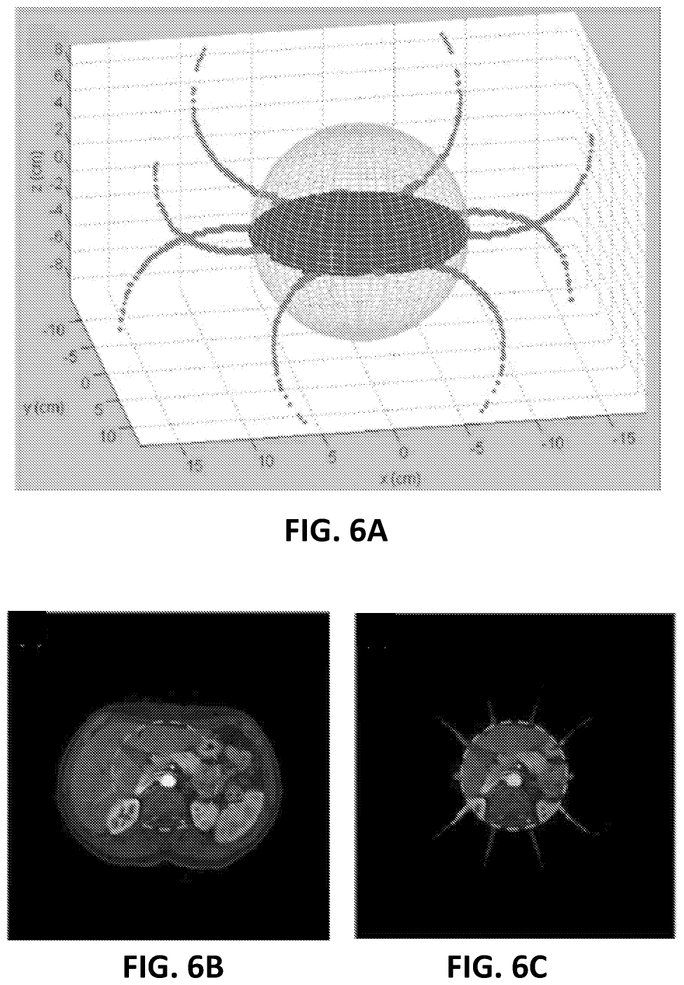

To demonstrate the principle of data acquisition, an assumed z gradient field with G_{z}=30 mT/m was applied, and an RF pulse corresponding to the slice at z₀=0 was used to select this slice. As mentioned above, a set covering this slice inside the ROI, as well as many other points outside the ROI would be selected. This is clearly demonstrated in FIG. 6A. Those dark voxels are selected voxels inside the 15 cm-diameter ROI that is indicated by the sphere. The voxels formed a slice as expected. In addition, a number of voxels outside the ROI (dark) were also selected.

Because of the magnetic field distribution, the selected voxel outside the ROI did not fall back to the disk region corresponding to the selected slice. This property ensured that the excited signal \hat{M}_{(x,y)}=∫_{Ω(x,y)}dzM(x,y, z) is identical to the expected signal M(x,y,z₀) inside the ROI. To see this fact more closely, FIG. 6B displays the true image \hat{M}(x,y,z₀) at the slice z₀, whereas the actual excited signal corresponding to an image \hat{M}_{(x,y)} shown in FIG. 6C. Inside the ROI indicated by the circle, the image \hat{M}_{(x,y)} corresponds to the actual image. In contrast, the part outside the ROI comes from other locations in the 3D space, not even in the slice z=z₀.

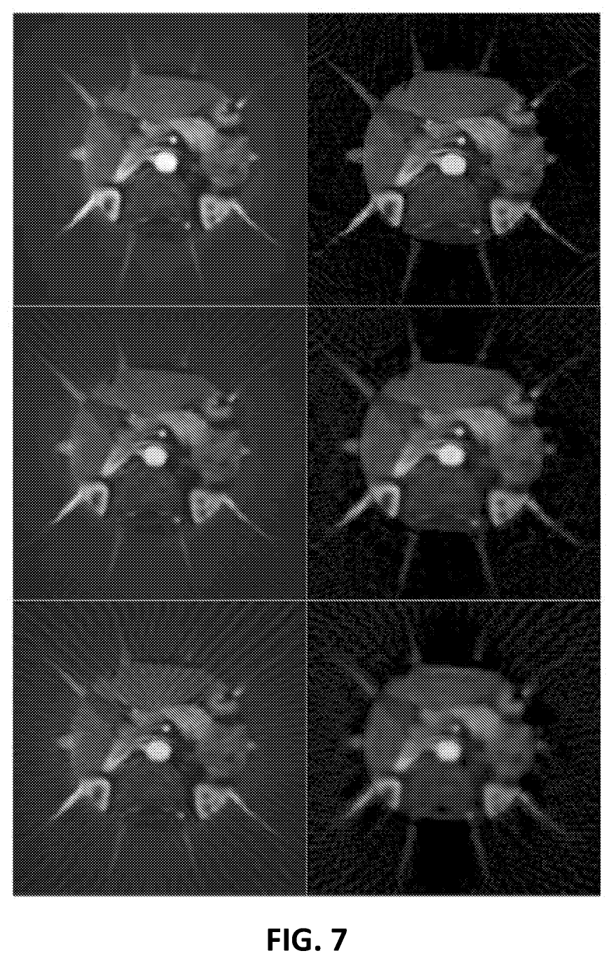

With the excitation signal generated, reconstruction was performed using the model in Eq. (3), as well as the conventional FBP algorithm for a comparison purpose. FIG. 7 shows the reconstruction results using conventional FBP (left) and method described herein (right) using 360, 180, and 120 projections respectively. With a large number of 360 projections acquired, both the FBP and the iterative algorithm were able to produce high quality images. Again, only the part within the central circular region is of interest, whereas the part outside should be ignored. When it comes to undersampling cases, streak artifacts start to appear in the FBP results, which is known for analytical reconstruction algorithms. In contrast, the iterative reconstruction algorithm was still able to maintain image quality to a good extent.

First, the studies shown illustrate the function of an iMRI, but have not been optimized. The main magnetic field B₀, can be designed through an optimization approach to yield the targeted strength and homogeneity, while maintaining the field strength at the LINAC gantry head to a tolerable level. Yet the field may violate other constraints posed by the LINAC. Hence, further field optimization may be needed, e.g. to reduce periphery field strength. A certain type of magnetic shielding to further reduce periphery field strength and therefore minimize interference with the LINAC can be implemented. Other factors, such as the impact of multi-leaf-collimator motion, if the iMRI device will be used for imaging during IMRT treatment delivery have been considered, but not tested.

The signal excited by the RF pulse comes from both inside the ROI and some regions outside. While this seems to be not a problem in the current study, it posed a challenge in the main magnetic field design: for each slice selected, the set should not contain the part inside the disk region but in other z slices. Otherwise the signal would be picked up by the excitation, and hence compromising the targeted images inside the ROI. There are other possible approaches to overcome this problem, such as a time-varying gradient method. The gradient field effectively suppresses signal excitations outside the ROI. In the context of parallel MRI, the use of multiple receiving coils with different spatial sensitive maps may also add additional information to differentiate the true image inside the ROI and that outside.

Recently, a few exciting achievements in the area of compact MRI scanner were reported, which demonstrated the great potential to develop lightweight and LINAC-compatible MRI systems. One notable example is the development from MIT that realized 2D imaging capability in a portable MRI scanner of <100 kg in weight. With a rotating spatial encoding method, the system eliminated the needs of gradient coils, substantially reducing system weight and complexity. Extending to 3D imaging capability is under exploration. A system design similar to this is potentially suitable for the integration to the LINAC platform. In the interior tomography framework, a complementary CT image is needed for global imaging, which utilizes a rotational scan. Hence combining the CT and the MRI data acquisitions in a single gantry rotation is a natural choice. For example, a similar idea has also been proposed in a recent study regarding the combination of CT and MRI systems.

This choice, however, limits the system to acquire data only at a zero-degree LINAC couch angle. This fact leads to both advantages and drawbacks. The advantage is relaxed constraint on geometry conflicts between MRI and CT sub-systems. Since the rotational data acquisition has to be performed at zero degree couch angle, geometry constraints with non-zero couch angle setups do not need to be considered anymore. The lightweight system may also allow for a mobile design, which holds the iMRI device on a robotic arm. The device can be docked to the gantry for pre-treatment imaging and removed for treatment delivery. In this way, the non-coplanar treatment capabilities, particularly 4π treatment capability on the current LINAC will not be affected. On the flip side, one drawback of this approach is that 4D imaging during treatment delivery will not be available due to the rotational data acquisition. Yet the necessity of this function in radiotherapy depends on specific clinical applications. While it is desired to monitor tumor and anatomy motion during a treatment via an imaging approach, using pretreatment MRI image guidance can already ensure targeting accuracy to a large extent. This would be already a significant step forward over the current CBCT-based pre-treatment image guidance. The residual intra-fractional motion can be addressed by using a treatment planning margin, as in the current standard approach.

Another concept for MRI-based image guided radiation therapy via an interior tomography approach can be used including an iMRI device design and integrated to a LINAC platform. The iMRI device can be made compact, such that it can be retrofit to an existing LINAC system to allow MRI-guided radiation therapy. A few aspects of the system were studied via numerical simulation, including main magnetic field design, signal acquisition, and image reconstruction. The image results were shown as an example. The system may hold a significant cutting-edge impact over the competing systems in terms of cost, functionality, and potential for clinical translation. Clinical introduction of the iMRI system may lead to a profound healthcare impact on cancer treatment by substantially improving treatment accuracy under the MRI-based image guidance.

Using a similar approach, an embodiment of an MRI adaptation can be configured for a Retrofit MRI (RMRI) assembly to mount on existing circular or “O-Ring” radiation therapy machines used in cancer treatments to provide MRI guided radiation therapy or MRgRT. Examples of different circular radiation therapy machines include LINAC (linear accelerator; radiation produced from accelerated electrons) and gamma knife (radiation produced from radioactive sources). A RMRI assembly can also be configured to be mounted on existing proton beam, heavy ion, electron cancer therapy machines, and the like.

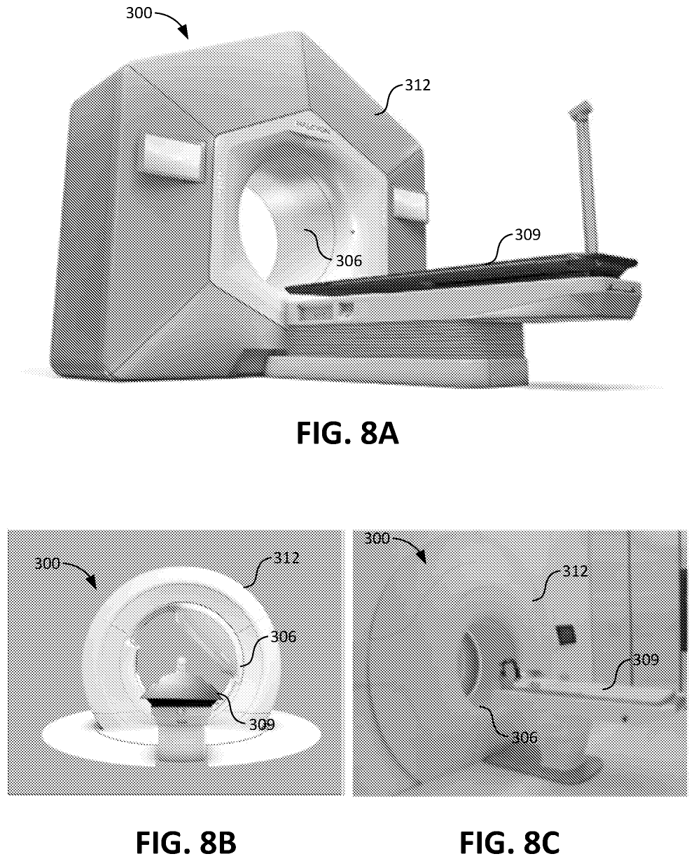

As an example, this disclosure describes the methods and systems for an RMRI configured for a radiative therapy LINAC machine, but the methods carry over to the other circular therapy machines, by taking into account the geometry of the other machines. Examples of different existing circular LINAC machines 300 available from different manufactures are shown in FIGS. 8A-8C, such as those manufactured by Accuray, Varian and BrainLab. In addition to circular LINAC machines, circular proton, heavy ion, electron or other photon (radiation) cancer therapy machines can also mount a Retrofit MRI assembly. The various example methods described in this application may be extended to the individual geometries of the machines of the different companies.

For example, a LINAC machine in FIG. 8A has circular ring gantry 303 (not shown) about a main bore 306. The patient 10 can be positioned on a movable couch or table 309, which can then be positioned within the bore 306 for radiotherapy. In this type of configuration, an outer shell 312 of the LINAC 300, can be removed to provide access to the main system components of the circular radiation machine.

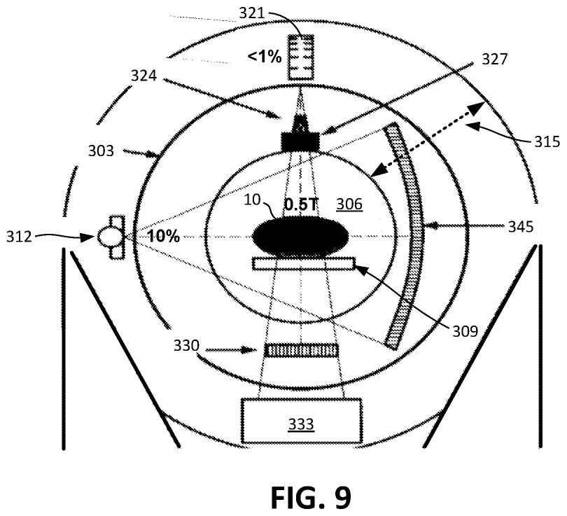

As shown in FIG. 9, in some embodiments, the main system components of a LINAC machine 300 are housed in a region 315 about the circular ring gantry 303 behind the removable shell 312 of the LINAC machine 300. In this example, the LINAC machine 300 can have treatment components 318 comprising an in-line LINAC 321 with a temporally modulated multileaf collimator (MLC) 324, a slit defining secondary collimator 327, megavoltage image detectors 330, and a beam stop 333 provide radiotherapy to a patient 10. The in-line LINAC 321 accelerates charged particles that collide with a target to produce photons or radiation for treatment. The circular LINAC machine 300, can also house a CT imaging system 339 positioned perpendicular to the LINAC treatment components 318. The CT imaging system 339 can comprise a CT x-ray source 342 and CT image detectors 345, each mounted on the ring gantry 303 on opposite sides to provide CBCT images of the patient 10 for proper positioning for radiotherapy.

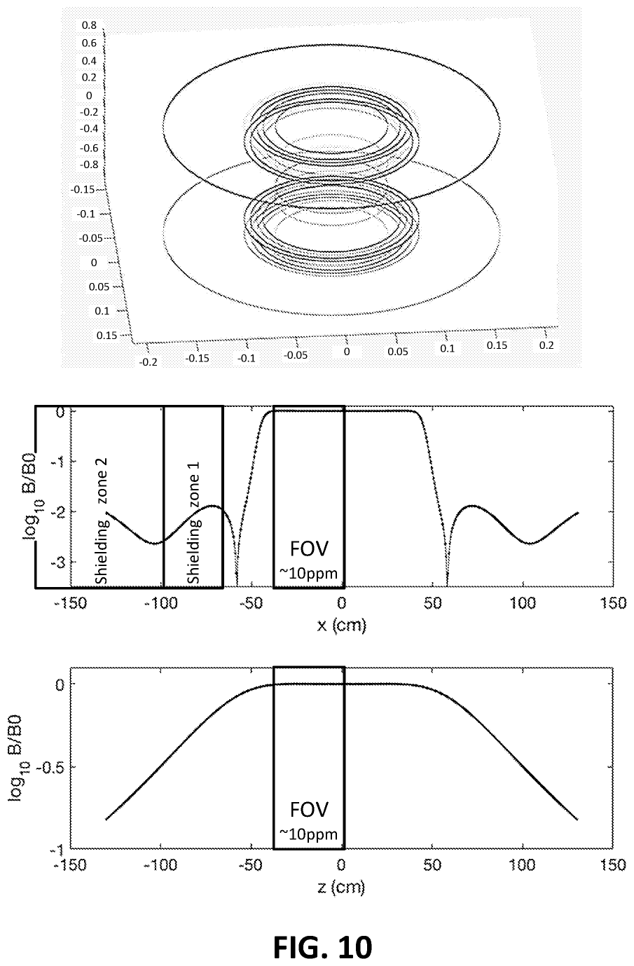

Unlike a conventional MRI system which employs a too bulky and complex design to realize a sufficiently large field of view (FOV), the RMRI can achieve uniform magnetic field (e.g. 0.4-0.7 T) over 30-50 centimeters. As shown in FIG. 10, an example desired magnetic field intensity is shown providing uniformity within a 40 cm diameter field of view at the center of an example existing circular radiation therapy machine 400. The field is generated by a main magnet using a superconducting magnet and uniformity of the magnetic field in the Field of View is a design goal.

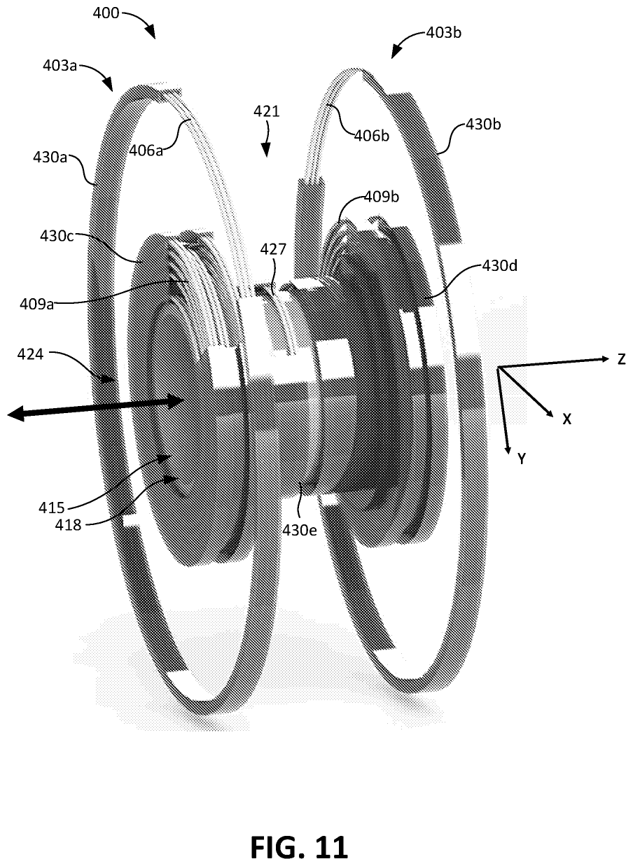

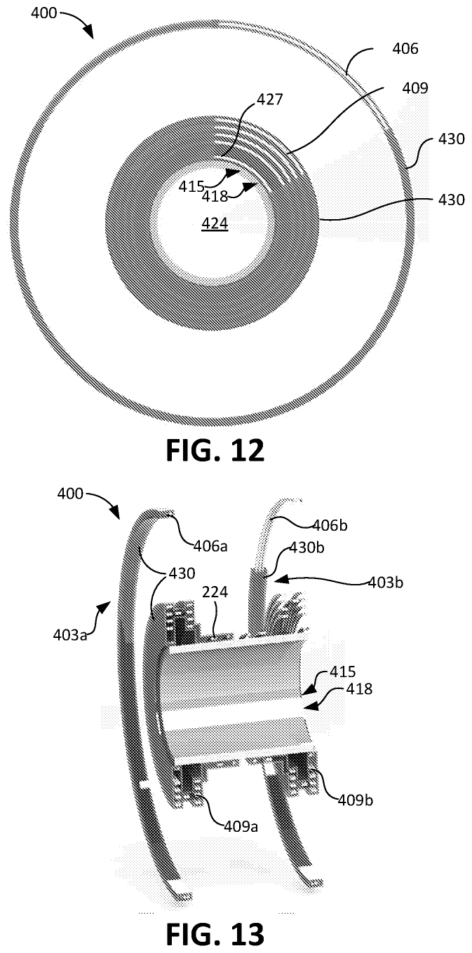

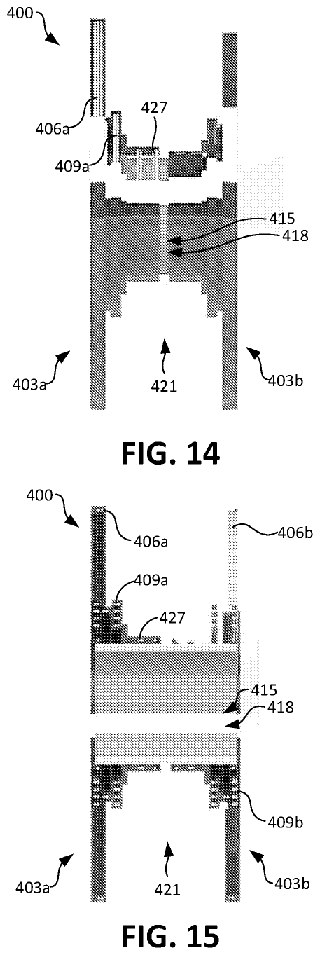

The RMRI assembly 400 can be configured to be housed within the spatial limitations of a circular radiation therapy machine, such as the LINAC machine 300. As shown in FIGS. 11-15, the RMRI assembly 400 includes a main magnet 403 having high temperature superconducting coils 406,409, a gradient field generation system 412, shimming system 415, an RF system having sender and receiver coils 418, and other subsystems (e.g. shielding for the LINAC). An RMRI assembly 400 has various design and mounting challenges (e.g. encountering pre-existing geometries and materials that cannot be changed), but when these challenges are overcome, a low-cost solution is achieved and pre-existing circular radiation therapy machines benefit from getting improved contrast image scans of the patient's anatomical and tissue features.

The example Retrofit MRI assembly 400 generates a magnetic field in a way to create an image scanner on an existing circular radiation therapy machine 300, such as a LINAC 321 that accelerates charged particles that collide with a target to produce photons or radiation. For example, when considering an existing circular design of FIG. 9, the magnetic field intensity can be strong enough (e.g. >0.5 Tesla) at the site of the patient to affect the proton spins of the water molecules in a patient, to produce radio (RF) signals that are measured by RF receivers and made into an image. In the example design, the magnetic field intensity reduces (see FIG. 9) down to about 1% (0.005T) away from the center of the gantry ring or O-ring so as to avoid disturbing the charged particles in the LINAC. This is a challenging engineering problem because of the sharp conflict between the RMRI assembly and the LINAC/circular radiation therapy machines in terms of electromagnetic physics and physical geometry. For example, a strong magnetic field used by the RMRI affects many electronic components inside the LINAC and circular radiation therapy machine that are susceptible to electromagnetic interference.

Turning to FIG. 11, shown are the main components of an example Retrofit MRI assembly 400. For description purposes, the Retrofit MRI 400 has a “yo-yo” configuration about a hollow cylinder 421 with a geometric isocenter 424, where the main magnet 403 is split into two portions 403a,403b similar to the side discs of a yo-yo. The hollow cylinder 421 configured to surround a patient tunnel or bore 306 of an existing circular radiation therapy machine 300. For example main magnet 403 comprises first superconducting magnet 403a having two main groups of concentric circular super conducting wires 406a,409a and second superconducting magnet 403b having two main groups of concentric circular super conducting wires 406b,409b. The two superconducting magnets 403a,403b comprising the four main groups of superconducting wires 406a,409a,406b,409b sandwich an additional group of concentric circular superconducting wires (fifth group) 427 disposed about the hollow cylinder 421. Each of the five groups of superconducting wires 406a,409a,406b,409b,427 are surrounded by a respective cryostat housing 430 to cool the superconducting wires inside the cryostat housing 430. Additionally, disposed within the innermost region of the hollow cylinder 203 and concentric with the outer main magnet coils 209,215,218,221, there are shimming coils 415 and gradient coils 418. Example superconducting wires can be made by STI having 600 A copper HTS (70 μm×12000 μm) @ 77° K.

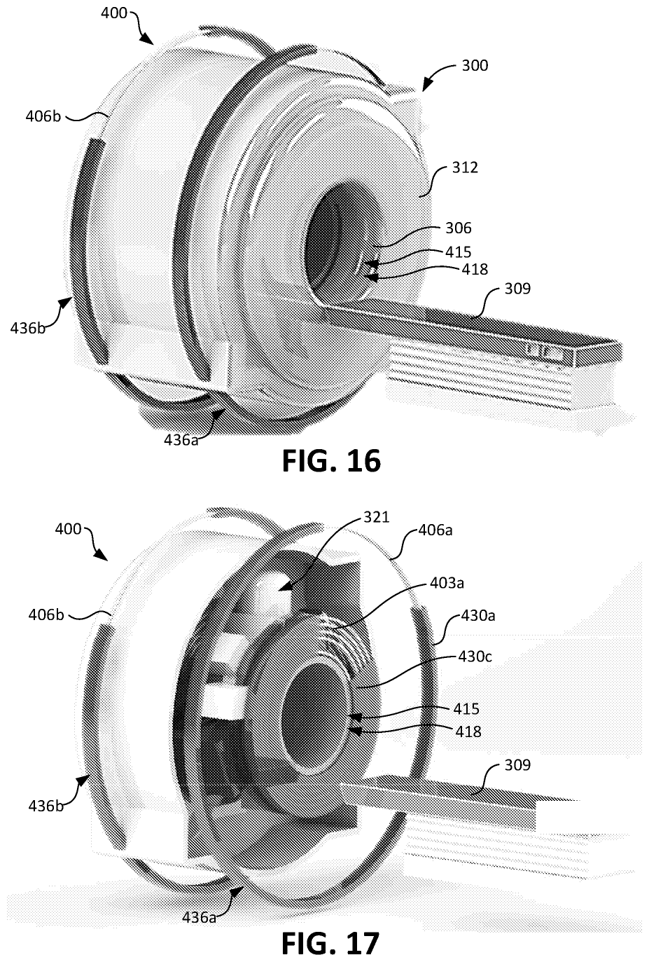

The example RMRI assembly 400 of FIG. 11 can be mounted to existing radiation therapy machines in an example manner as shown in FIG. 16, which represent an example LINAC machine 300 as shown in FIG. 8 with the shell 312 removed. FIG. 17 shows a cutaway view of the installation of FIG. 8. The example main magnet 403 includes two groups of mounting rings 436a,436b. The mounting rings 436 comprise cryostat housing 430 for superconducting wires 406 installed on an exterior of an existing gantry 303 of a circular radiation therapy machine 303 and concentric about an isocenter 424 of the gantry 303; a first set of superconducting wires 406a is positioned external to one end of the gantry; a second set of superconducting wires 406b is positioned external to the opposite end of the gantry 303. The first and second sets 406a,406b are separated by the gantry width W (along the cylinder). The first and second set of wires 406a,406b are parallel to each other and help create a main magnetic field within a region of interest 439, the region of interest located at an isocenter 424 of the gantry 303.

The main magnet 403 can be split into two portions as configured to address the geometric and electromagnetic limitations of the existing circular radiation therapy machine 300. A first superconducting magnet 403a includes a first set of superconducting wires 406a and additional superconducting wires 409a concentric to, but at a smaller radius than the first set of superconducting wires 406a. Similarly, a second superconducting magnet 403b includes a second set of superconducting wires 406b and additional superconducting wires 409b concentric to but at a smaller radius than the second set of superconducting wires. There is another set of superconducting wires 427 at the center of the “yo-yo” configuration (see FIG. 11) sandwiched between the two additional sets of superconducting wires. Stated another way, there is another set of superconducting wires 427 at the center between the two superconducting magnets 403a,403b.

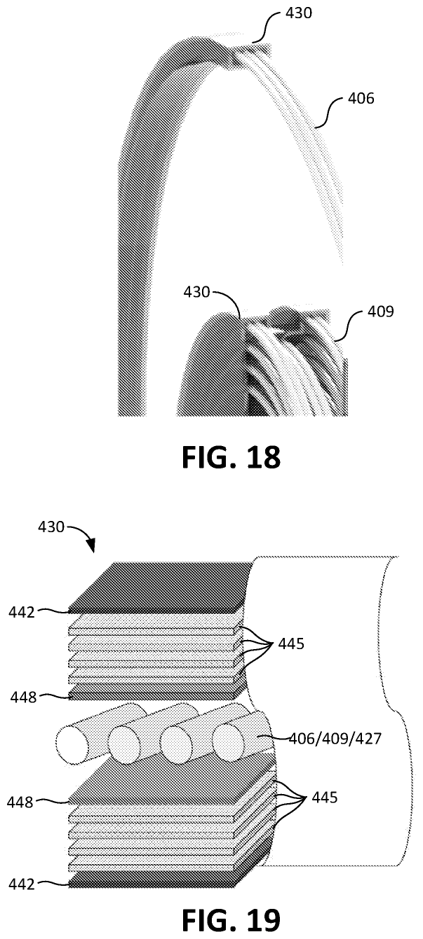

Next, FIG. 18 shows an enlarged view of the superconducting wires 406a and 409a and with a respective cryostat housing 430a, 430b of magnet 403a of FIG. 11. Shown in this example, the cryostat housing 430 is configured to provide cooling to superconducting wires 406a,406b; however, similar housing is sized and shape for each implementation of superconducting wires 406a,406b,409a,409b,427.

Shown in FIG. 19 is an illustration of layers of the cryostat housing 430 to house superconducting wires 406a,406b,409a,409b,427. The cryostat housing 430 comprises an outer steel wall 442, a plurality of reflector layers 445, and an inner steel wall 448. The cryostat housing can be configured to accommodate superconducting wires 406a,406b,409a,409b,427 at respective coil locations and configurations.

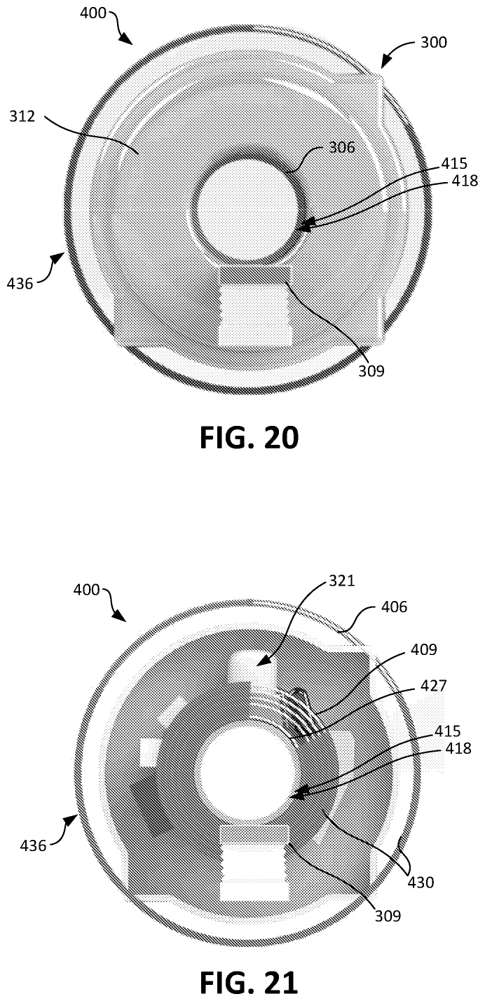

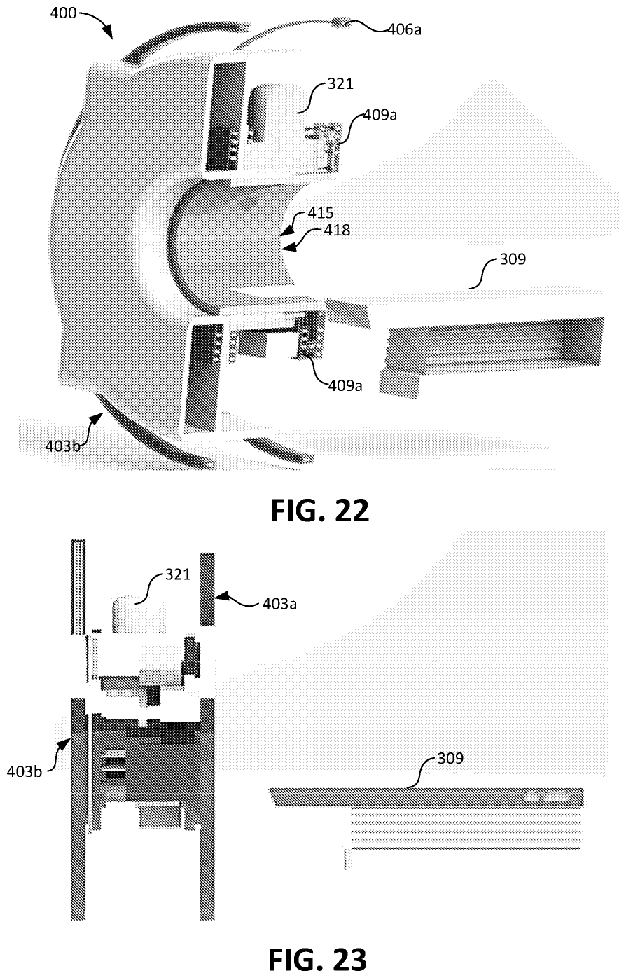

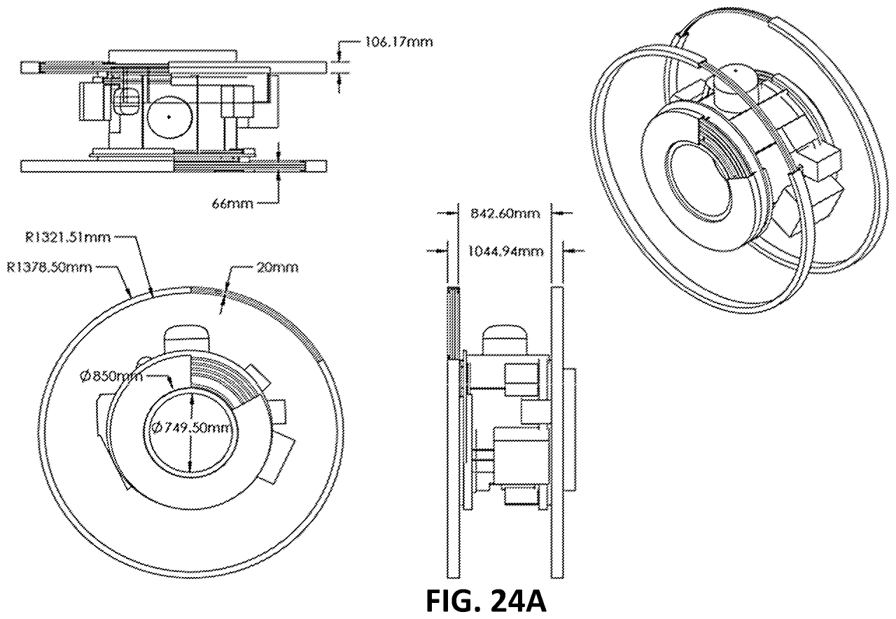

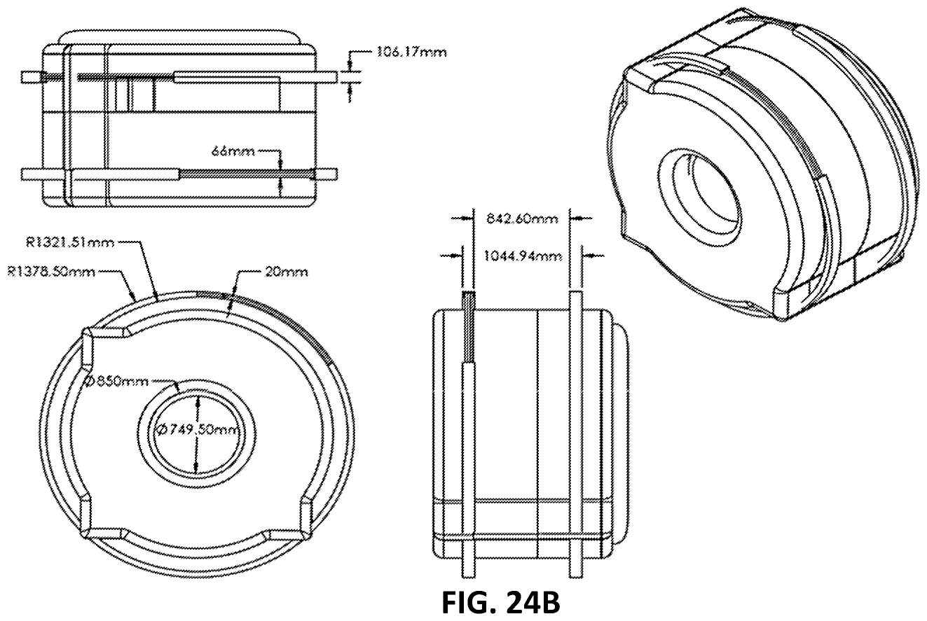

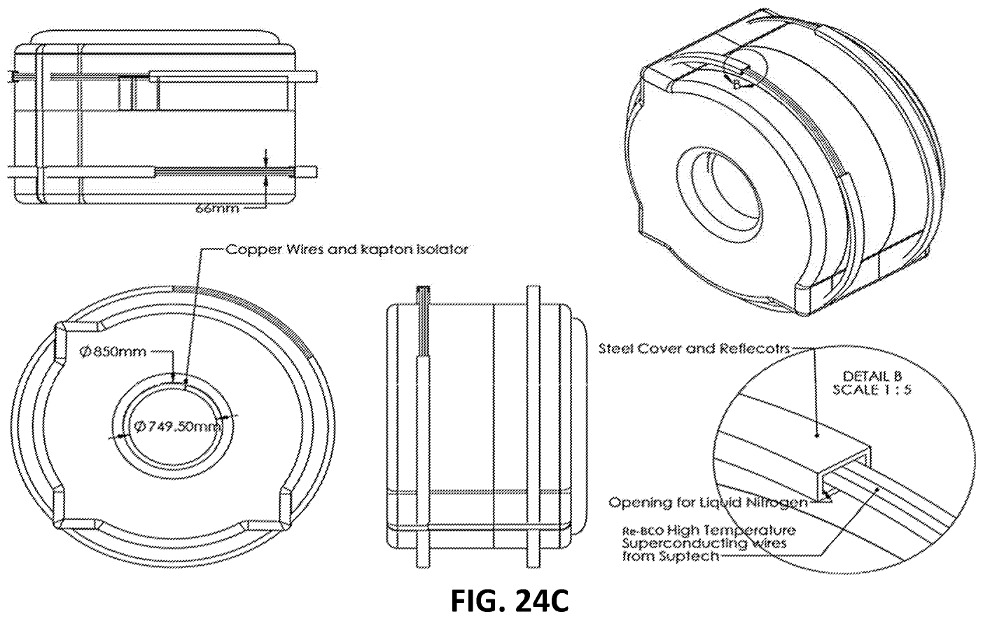

To further illustrate the RMRI assembly 400 in an example installation, FIGS. 20-23 illustrate various views of the example Retrofit MRI assembly mounted on the example existing circular radiation therapy LINAC machine of FIG. 16. FIGS. 24A-24C include the mechanical details of different example components of a Retrofit MRI assembly. The dimensions and materials are shown in the FIGS. 24A-24C are for dimensional illustration purposes only, as the RMRI assembly 400 can be configured to meet the requirements of a specific circular radiation therapy machine 300.

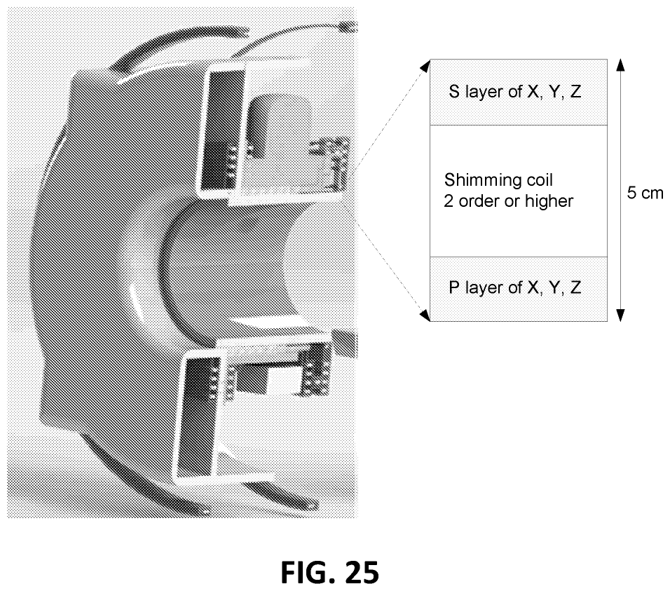

FIG. 25 illustrates an example gradient field generator used in the example Retrofit MRI assemblies. The gradient field generator comprises six layers of self-shielded gradient coils and six layers of shimming coils. A gradient coil contains two layers. The primary layer (p layer) is used to generate the targeted gradient field, and the shielding layer (s layer) is used to shield the gradient magnetic field outside the gradient coils. The shimming coils contain a number of sub-coils each can produce a magnetic field with a spatial variation described by a 2^{nd} order polynomial, e.g. xy, xz, yz. Together, these coils can be used to generate a magnetic field described by any 2^{nd} order polynomial for shimming purpose.

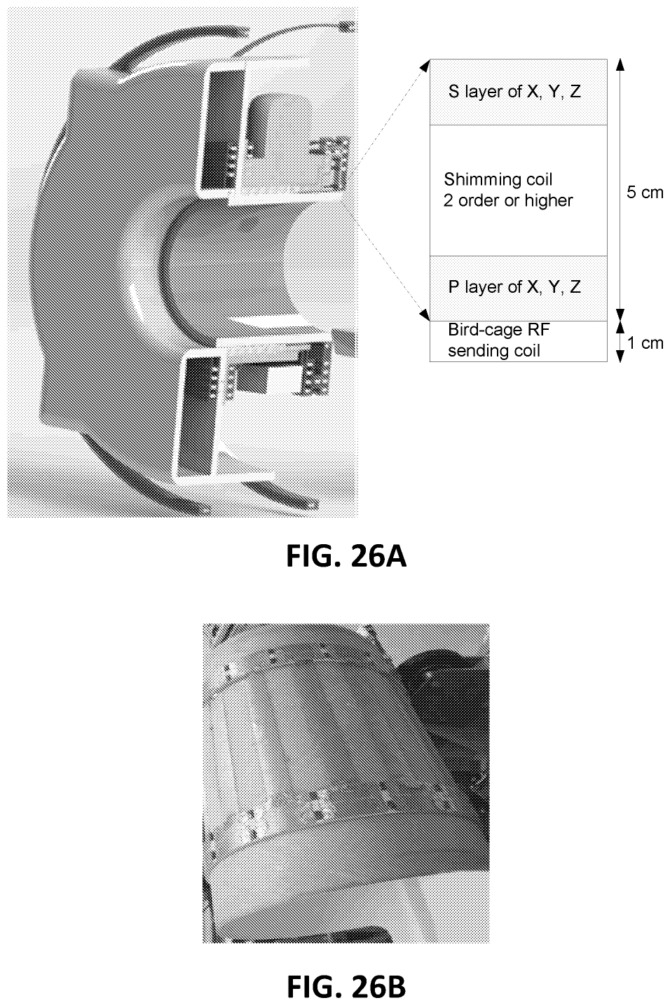

In an embodiment, a bird-cage RF sending coil can be included as shown in FIG. 26A. Illustrated in FIG. 26B is an example RF sender coils used in the example Retrofit MRI assemblies.

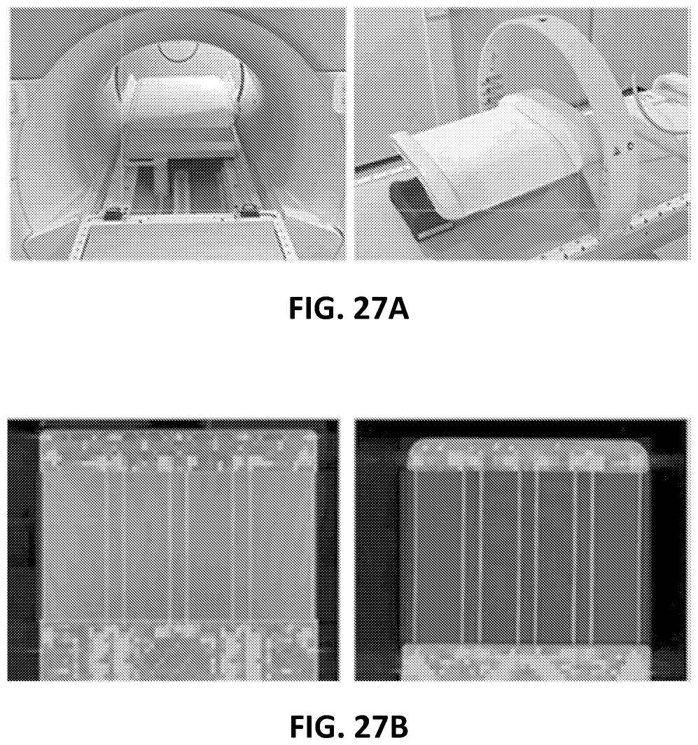

FIG. 27A illustrates an example of already-available RF receiver coils. Shown is an MR coil having anterior and posterior segments mounted to the patent couch. There are MR (magnetic resonance) coils around a patient; the MR coil includes anterior and posterior segments in an exposed treatment position (table moved out of position). An example anterior coil is mounted on the ring in order to be placed above the patient. The posterior segment is placed 7-10 mm beneath a treatment table. Shown in FIG. 27B is a maximum intensity projection of each segment made of radio-translucent ribbons in the middle.

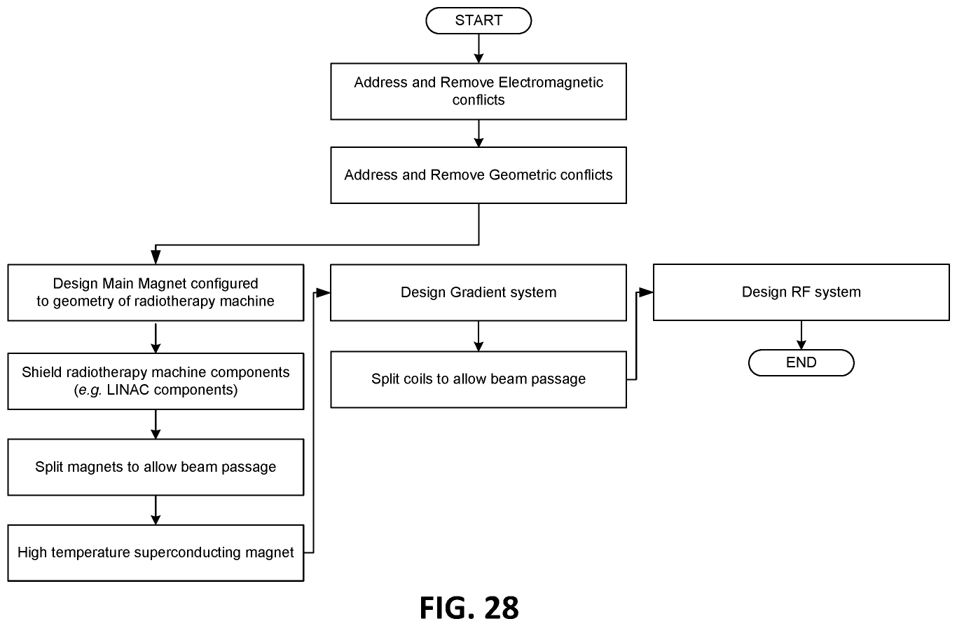

FIG. 28 includes an example method and problem issues addressed when designing or making a Retrofit MRI assembly for different geometries of pre-existing circular radiation therapy LINAC machines. For instance, the main magnetic field B₀, is designed through an optimization approach to yield the targeted strength and homogeneity, while maintaining the field strength at the LINAC gantry head to a tolerable level. Hence, further field optimization may be needed, e.g. to reduce periphery field strength. A certain type of magnetic shielding to further reduce periphery field strength and therefore minimize interference with the LINAC can be implemented. Other factors, such as the impact of multi-leaf-collimator motion, if the RMRI device will be used for imaging during IMRT treatment delivery have been considered.

For a specific example in the design of main magnetic field, one uses a few wires (e.g. 3-5) that carry superconducting currents circulating in planes parallel to the O-Ring LINAC rotational plane. Superposition of magnetic fields created by these currents yields the magnetic field used for MR imaging such as the values shown in FIGS. 9 and 10. The design includes a homogeneous magnetic field of at least 0.5 T with a homogeneity level of <10 ppm inside the imaging region of interest (ROI) or field of view (FOV), e.g. a spherical region of diameter of 35-45 cm.

In addition, the magnetic field reduces in the region covering a ring gantry region containing the LINAC parts that are magnetically sensitive (see FIG. 9). An example targeted magnetic field is, e.g. <50 Gauss. Although this field may not be low enough to ensure normal functions of LINAC components, with some local shielding (e.g. passive materials or metal such as steel), the field at sensitive components is reduced to a safe level to ensure LINAC functionality in a transition region between the imaging ROI and the low field donut region.

The goals of the design method further include first identifying candidate spaces that are to be used to house superconducting currents and associated cooling components. A candidate space is selected to be geometrically compatible with the O-ring LINAC, e.g. components in the space that do not interfere with rotation of the LINAC and also do not block radiation beams.

The design further includes computing a magnetic field distribution Bᵢ (x) of each candidate superconducting current i at its unit current intensity, where x is the spatial coordinate. The total magnetic field is:

B(x)=ΣᵢBᵢ(x)Iᵢ

The example design further involves solving an optimization problem with respect to Iᵢ subject to the constraints specified by the desired field distribution. A sparsity term of the currents was used to ensure a small number of currents in the solution. The solution yielded the current pattern generating the desired target magnetic field distribution. Alternatively, this process is achieved by using a different representation of candidate currents, such as currents with a rectangular cross section or current sheets.

To further consider the effects of perturbations of surrounding magnetic materials on the magnetic field generated, the design method includes calculating the perturbation using a finite-element solver. The perturbation is then considered in the optimization process to generate an updated current solution. This process is iterated until convergence is reached.

The example design method for the gradient coils and shimming coils yields several cylindrical surfaces located close to the LINAC bore surface. Next, three pairs of coils are considered to generate magnetic fields that linearly vary along x, y, and z direction. Each contains a pair of coils with currents flowing on two cylindrical surfaces. Superposition of the magnetic fields created by the pair of coils yields the targeted linear magnetic field inside the FOV while a shield field is implemented in nearby metallic components to reduce eddy currents. The method represents each layer with a sheet current using stream functions. Further, the layers were designed to have an opening to allow radiation beam to pass through. An optimization problem was solved with respect to the stream functions to generate the desired gradient fields. The stream function was then converted into current pattern by drawing iso-contour lines.

The shimming coil design follows a similar approach as the gradient coil design. There are a number of layers on cylindrical surfaces with different radii. The current on each layer generates a magnetic field that varies in a high-order polynomial form in the imaging FOV. After designing the position of each layer with an opening to allow radiation beam to pass through, the stream function optimization method was used to determine current pattern that creates the targeted magnetic field.

An example design of the RF components includes a birdcage-design for the sending or transmitting RF coil. The coil located on a cylindrical surface interior to the gradient and the shimming coil. A birdcage-design with two end rings and multiple (e.g. 16) rungs is used. An example receiving RF coil includes a surface coil, and surface coil array. In one example, the RF coil is placed in front of the patient body, on the side of the patient body, or embedded in the LINAC couch. The birdcage sending/transmitting RF coil can also serve as the receiving coil.

The following describes various embodiments of the operation of the RMRI embodiments as shown in FIGS. 11-27.

For example, the RMRI system performs data acquisition, taking into account the homogeneous main field B₀ that exists only in a relative small ROI region (e.g. 40 cm). A slice selection technique is used by applying a slice selection z-gradient field. The other two gradient fields will be used for frequency encoding and phase encoding. For example, considering a slice orthogonal to the z axis with a coordinate z=z₀, a radio frequency (RF) pulse with frequency f₀=γ(B₀+z₀G_{z}) should be used, where G_{z} is the gradient amplitude. At the moment of measurement, the signal after demodulation is

s(kₓ, k_{y})=∫dxdy M(x,y,z₀)e^{-i(k}^{ₓ}^{x+k}^{_{y}}^{y)} (4)

where M(x,y,z) is the 3D magnetization distribution. With the measurement S(kₓ, k_{y}) made, standard reconstruction techniques using analytical reconstruction or iterative reconstruction techniques apply. For example, the conventional Fourier Transform based reconstruction method can be used to solve M(x, y).

For radiotherapy online imaging applications, data acquisition time is also optimized. Therefore, k-space data undersampling is desired to speed up the data acquisition process. In this case, iterative reconstruction will be advantageous.

Below, the magnetization is represented by a vector u. Discretizing Eq. (4) arrives at a linear equation:

AFu=g, (5)

where F is the Fourier transform operator, A is an undersampling operator corresponding to the sampled k-space locations, and g is a vector containing the measurement data. Since the solution u represents a 2D image, a certain type of image regularization can be applied to constrain the solution. As an example, tight frame (TF) can be used as a regularization transformation and solve the problem

$${min}_{u} {\vert Wu \vert}_{1} , s . t . AFu = ℊ , \qquad ( 6 )$$

where W is a TF transform operator. Minimizing the I₁ norm of the transformed image Wu assumed that the solution image u has a sparse representation under the TF transformation. This optimization problem (3) can be efficiently solved using the alternating direction method of multipliers.

Simulation studies were performed to further demonstrate a magnet design. For example, an inverse optimization problem was solved with respect to the current pattern inside the superconducting magnets. One objective function penalized deviation of the magnetic field from the targeted homogeneous field B₀=0.5 T throughout the FOV. A constraint is also imposed to ensure the field strength at the LINAC gantry head is tolerable. After that, a volumetric MRI image of a cancer patient was selected. RF signal propagation and interaction with the patient was simulated using Bloch equation.

With the synthesized data, the image is then reconstructed via the model in Eq. (6). For comparison purpose, reconstruction using the conventional Fourier Transform algorithm was also performed. The image quality is evaluated by comparing with the ground truth input MRI image. The Retrofit MRI assembly may perform compatibly with any other image reconstruction approaches.

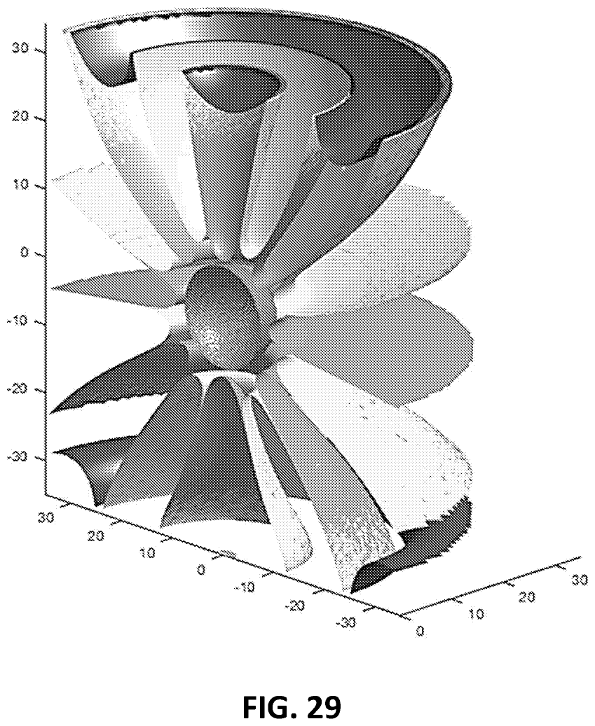

FIG. 29 presents the spatial distribution of the x and y component of the B₀ field in the x-y plane. The field component perpendicular to this plane is zero. The field in 3D space is rotationally symmetric about the y axis. Using the current optimization technique, the B₀ field was made homogeneous inside the FOV of a diameter of 40 cm with ΔB₀/B₀ about 10 ppm. In addition, at the positive direction y about 85 cm locates the gantry head of a LINAC. The field at this location was constrained to be less than a threshold value that is expected to be tolerable by a LINAC.

For example embodiments of the data acquisition, a z gradient field is assumed with G_{z}=some value (e.g. 30 mT/m) applied, and an RF pulse corresponding to the slice at z₀=0 was used to select this slice.

With the excitation signal generated, reconstruction was performed using the model in Eq. (6), as well as the conventional Fourier Transform algorithm. With a large number of 360 projections acquired, both the Fourier Transform and the iterative algorithm were able to produce high quality images.

In another data acquisition embodiment, 3D imaging capability is realized based on a 2D imaging capability in a portable MRI scanner of <100 kg in weight. With a rotating spatial encoding method, the system eliminated the needs of gradient coils, substantially reducing system weight and complexity. This is to combine the CT and the MRI data acquisitions in a single gantry rotation.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt% to about 5 wt%, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

We claim:

1. A system for MRI-guided radiotherapy, comprising:
a mounting ring configured to be installed on a gantry of a linear accelerator (LINAC) and configured to rotate about an isocenter of the LINAC moving with the gantry;
a first superconducting magnet connected to the mounting ring, the first superconducting magnet positioned in a first plane contacting a gantry head of the LINAC;
a second superconducting magnet connected to the mounting ring, the second superconducting magnet positioned in a second plane substantially parallel to the first plane at a separation distance, a center of the second superconducting magnet substantially aligned with a center of the first superconducting magnet;
wherein the first and second superconducting magnets are configured to provide a main magnetic field within a region of interest, the region of interest located between the first superconducting magnet and the second superconducting magnet; and
wherein each of the first and second superconducting magnets have an aperture between an inner surface facing the isocenter and an outer surface facing away from the isocenter, each aperture positioned at the center of each superconducting magnet and configured to allow a radiotherapy beam emitting from the gantry head to pass through the apertures.

2. The system of claim 1, wherein the main magnetic field produced by the first and second superconducting magnets do not substantially interfere with operation of the LINAC.

3. The system of claim 1, wherein a measurement of magnetic field near an accelerating waveguide of the LINAC is less than 5 Gauss.

4. The system of claim 1, wherein a measurement of magnetic field near the gantry head of the LINAC is less than 400 Gauss.

5. The system of claim 1, wherein a measurement of homogeneity (ΔB₀/B₀) for magnetic field within the region of interest is less than 50 ppm.

6. The system of claim 1, wherein the gantry head of the LINAC is shielded from magnetic fields.

7. The system of claim 1, wherein the mounting ring is further configured to provide shielding from at least one of: magnetic fields, x-rays, or photons produced by the LINAC.

8. The system of claim 1, further comprising a magnetic resonance detector configured to collect excitation signal data to generate a magnetic resonance image.

9. The system of claim 8, wherein the mounting ring is further configured to mount an x-ray imaging system, the x-ray imaging system configured to operate in a plane perpendicular to the radiotherapy beam of the LINAC, the x-ray imaging system comprising:
an x-ray source attached to the mounting ring and configured to direct an x-ray beam toward the region of interest; and
an x-ray detector attached to the mounting ring opposite the x-ray source, and
wherein the magnetic fields produced by the first and second superconducting magnets do not interfere with operation of the x-ray imaging system.

10. The system of claim 9, wherein the x-ray detector is configured to collect x-ray data to generate an x-ray tomographic image of the region of interest.

11. The system of claim 10, wherein the x-ray data and the excitation signal data are collected simultaneously with rotation of the LINAC gantry.

12. The system of claim 1, wherein the region of interest is within a body of a patient, the patient being positioned on a patient couch to receive radiotherapy treatment.

13. The system of claim 1, further comprising:
at least one computing device; and
program instructions executable in the at least one computing device that, when executed by the at least one computing device, cause the at least one computing device to:
collect data from excitation signals to generate a magnetic resonance image containing the region of interest; and
apply a regularization transformation to portion of image containing the region of interest.

14. A system for magnetic resonance imaging, comprising:
a first superconducting magnet positioned in a first plane; and
a second superconducting magnet positioned in a second plane substantially parallel to the first plane at a separation distance, a center of the second superconducting magnet substantially aligned with a center of the first superconducting magnet; and
wherein the first and second superconducting magnets are configured to provide a main magnetic field within a region of interest, the region of interest located between the first superconducting magnet and the second superconducting magnet.

15. The system of claim 14, wherein each of the first and second superconducting magnets comprise super conducting coils.

16. The system of claim 15, wherein each of the super conducting coils of the first and second superconducting magnets comprises a plurality of superconducting fibers, the plurality of superconducting fibers configured to receive liquid helium.

17. The system of claim 14, further comprising a plurality of coils configured to generate gradient magnetic fields along x, y, and z directions of an orthogonal coordinate system.

18. The system of claim 17, wherein the first and second superconducting magnets are positioned parallel to an x-z plane;
a first y-gradient coil positioned within the first superconducting magnet, the first y-gradient coil configured to provide a magnet field along the y-direction, the y-direction perpendicular to the x-z plane;
a first x-gradient coil positioned on an inner surface of the first superconducting magnet facing the region of interest, the first x-gradient coil configured to provide a magnet field along the x-direction; and
a first z-gradient coil positioned on the inner surface of the first superconducting magnet facing the region of interest, the first z-gradient coil configured to provide a magnet field along the z-direction.

19. The system of claim 14, wherein gradient data is collected to generate a volumetric image.

20. The system of claim 14, wherein each of the first and second superconducting magnets have an aperture between an inner surface facing the region of interest and an outer surface facing away from the region of interest, each aperture positioned at the center of each superconducting magnet and configured to allow a radiotherapy beam to pass through the apertures.

21. The system of claim 20, wherein the first superconducting magnet is positioned between a radiotherapy source and the region of interest and the second superconducting magnet is positioned between the region of interest and an imaging device.

22. The system of claim 14, wherein the region of interest is within a body of a patient.

23. The system of claim 14, wherein the main magnetic field (B₀) is approximately 0.2 to 0.8 Tesla within the region of interest.

24. The system of claim 14, wherein the separation distance is approximately 50 to 90 cm.

25. The system of claim 14, wherein the region of interest is within a substantially spherical region having a diameter of approximately 10 to 20 cm.

26. The system of claim 14, further comprising a magnetic resonance detector configured to collect excitation signals to produce an image.

27. The system of claim 14, further configured to be mounted on a robotic arm.

28. The system of claim 14, further configured to rotate about an axis.

29. A retrofit MRI assembly, comprising:
a main magnet comprising spatially separated first and second portions,
the first portion comprising a first set of superconducting wires disposed within a first circular housing concentric about an isocenter of a gantry of a circular radiation therapy machine, the first circular housing installed at a first end exterior to the gantry;
the second portion comprising a second set of superconducting wires disposed within a second circular housing concentric about the isocenter of the gantry, the second circular housing installed at a second end exterior to the gantry of the circular radiation therapy machine, where the second set of superconducting wires are substantially parallel to the first set of superconducting wires with a center of the second set of superconducting wires substantially aligned with a center of the first set of superconducting wires, the first and second sets of superconducting wires separated by at a gantry separation distance;
shimming coils; and
gradient coils;
wherein the first set and the second set of superconducting wires are configured to provide a main magnetic field within a region of interest located at the isocenter of the gantry.

30. The retrofit MRI assembly of claim 29, further including a third set of circular superconducting wires positioned at the first end of the gantry and a fourth set of circular superconducting wires positioned at the second end of the gantry, wherein the third set and the fourth set are concentric with the first set and the second set of superconducting wires, and wherein the third set and the fourth set are located at a smaller radii from the isocenter than the first set and the second set of superconducting wires.

31. The retrofit MRI assembly of claim 29, wherein the main magnet is configured to produce a magnetic field that does not interfere with an operation of a LINAC of the circular radiation therapy machine.

32. The retrofit MRI assembly of claim 29, wherein, when energized, a measurement of magnetic field near an accelerating waveguide of a LINAC of the circular radiation therapy machine is less than 5 Gauss.

33. The retrofit MRI assembly of claim 29, wherein, when energized, a measurement of magnetic field during operation near a gantry head of a LINAC is less than 400 Gauss.

34. The retrofit MRI assembly of claim 29, wherein a measurement of homogeneity (ΔB₀/B₀) for a magnetic field within the region of interest is less than 50 ppm.

35. The retrofit MRI assembly of claim 29, wherein a gantry head of a LINAC is shielded from magnetic fields.

36. The retrofit MRI assembly of claim 29, wherein the first circular housing comprise a cryostat housing and the second circular housing comprise a cryostat housing.

37. The retrofit MRI assembly of claim 29, further comprising a magnetic resonance detector configured to collect excitation signal data to generate a magnetic resonance image.

38. The retrofit MRI assembly of claim 29, wherein the circular radiation therapy machine comprises a LINAC and an x-ray imaging system, the x-ray imaging system configured to operate in a plane perpendicular to a radiotherapy beam of the LINAC.

39. The retrofit MRI assembly of claim 38, wherein the main magnetic field produced by the first and the second sets of superconducting wires does not interfere with operation of the x-ray imaging system.

40. The retrofit MRI assembly of claim 38, wherein the x-ray imaging system is configured to collect x-ray data to generate an x-ray tomographic image of the region of interest.

41. The retrofit MRI assembly of claim 29, further comprising:
at least one computing device; and
program instructions executable in the at least one computing device that, when executed by the at least one computing device, cause the at least one computing device to:
generate a magnetic resonance image containing a field of view based upon data collected from excitation signals; and
apply a regularization transformation to a portion of image containing the field of view.

42. A system for magnetic resonance imaging, comprising:
a first superconducting magnet positioned in a first plane; and
a second superconducting magnet positioned in a second plane substantially parallel to the first plane at a gantry separation distance, a center of the second superconducting magnet substantially aligned with a center of the first superconducting magnet; and
wherein the first and second superconducting magnets are configured to provide a main magnetic field within a region of interest, the region of interest located between the first superconducting magnet and the second superconducting magnet.

43. The system of claim 42, wherein each of the first and second superconducting magnets comprise super conducting coils.

44. The system of claim 43, wherein each of the super conducting coils of the first and second superconducting magnets comprises a plurality of superconducting fibers, the plurality of superconducting fibers configured to receive liquid helium.

45. The system of claim 42, further comprising a plurality of coils configured to generate gradient magnetic fields along x, y, and z directions of an orthogonal coordinate system.

46. The system of claim 45, further comprising a gradient field generator comprising a plurality of layers of self-shielded gradient coils and a plurality of layers of shimming coils.

47. The system of claim 45, wherein gradient data is collected to generate a volumetric image.

48. The system of claim 42, wherein the main magnetic field (B₀) is approximately 0.4-0.7 Tesla within a region of interest.
